(19) United States
(12) Patent Application Publication
(10) Pub. No.: US 2013/0060344 A1
(43) Pub. Date: Mar. 7, 2013

(54) FEMORAL COMPONENT WITH REINFORCED ARTICULATING SURFACE

(71) Applicant: Andrew Pierce, Warsaw, IN (US)

(72) Inventor: Andrew Pierce, Warsaw, IN (US)

(73) Assignee: BIOMET MANUFACTURING CORP., Warsaw, IN (US)

(21) Appl. No.: 13/227,159

(22) Filed: Sep. 7, 2011

(51) Int. Cl. A61F 2/38

(52) U.S. Cl. 623/20.28; 623/20.35

(57) ABSTRACT

A femoral component for a knee joint prosthesis. The femoral component can comprise a polymer body having first and second condylar portions interconnected by an intercondylar anterior portion. A first reinforcement member can be secured to the first condylar portion. A second reinforcement member can be secured to the second condylar portion. The reinforcement members can comprise a material selected from the group consisting of a metal alloy, a ceramic material, and a thermoplastic material.

### INTRODUCTION

The present technology generally relates to knee joint prostheses, and more particularly to knee joint components having reinforced articulating surfaces, and methods of their manufacture.

A knee joint prosthesis can generally comprise a femoral component and a tibial component. The femoral component and the tibial component can be designed to be surgically attached to the distal end of the femur and the proximal end of the tibia, respectively. The femoral component can further be designed to cooperate with the tibial component in simulating the articulating motion of an anatomical knee joint. In many examples, knee arthroplasty components have been fabricated with metal femoral and tibial components along with polyethylene bearings and polyethylene patellar buttons. While they provide high strength, the use of solid metal components leads to increased weight. Additionally, such metal components are typically made from casting techniques, which may require costly and time-consuming polishing and other treatments in order to provide a smooth exterior surface. Thus, in the growing field of knee arthroplasty and knee revision medical implants, there remains a need to provide more lightweight components while still maintaining and providing enhanced strength, especially at or near bearing surfaces and points of articulation.

### SUMMARY

The present technology provides a femoral component for a knee joint prosthesis. The femoral component can comprise a polymer body having first and second condylar portions interconnected by an intercondylar anterior portion. A first reinforcement member can be secured to the first condylar portion. A second reinforcement member can be secured to the second condylar portion. The reinforcement members can comprise a material selected from the group consisting of a metal alloy, a ceramic material, and a thermoplastic material.

The present technology also provides a knee joint prosthesis assembly. The assembly comprises a polymer femoral component including an intercondylar anterior portion. First and second condylar portions can extend from the intercondylar anterior portion. A first rigid reinforcement insert can be secured to the first condylar portion, defining a first reinforced bearing surface. A second rigid reinforcement insert can be secured to the second condylar portion, defining a second reinforced bearing surface. A tibial component can be provided having a bone engaging inferior surface and a bearing engaging superior surface configured to engage the first and second reinforced bearing surfaces of the polymer femoral component.

In another embodiment, a knee joint prosthesis is provided comprising a polymer femoral component including at least one of a lateral and medial condylar portion having a bearing surface. The bearing surface extends from an intercondylar anterior portion and defines an internal recess. A rigid reinforcement member can be disposed at least partially within the internal recess. A tibial component can be provided having bearing surfaces that are complementary to the respective condylar bearing surfaces of the polymer femoral component.

### DRAWINGS

FIG. 1 is an anterior perspective view of a knee prosthesis in accordance with various embodiments of the present teachings, with a tibia and femur illustrated in phantom;

FIG. 2 is an anterior view of an exemplary femoral component according to additional features;

FIG. 3 is a posterior view of the femoral component of FIG. 2;

FIG. 4 is a perspective posterior view of another femoral component according to additional features;

FIG. 5 is a cross sectional view of the femoral component of FIG. 4, taken along the line 5-5;

FIG. 6 is an anterior view of another femoral component according to additional features;

FIG. 7 is an anterior view of the femoral component of FIG. 6, including reinforcement members;

FIG. 8 is an anterior view of another femoral component according to additional features;

FIG. 9 is an anterior view of the femoral component of FIG. 8, including reinforcement members; and

FIG. 10 is an anterior view of another femoral component according to additional features.

It should be noted that the figures set forth herein are intended to exemplify the general characteristics of materials, methods and devices among those of the present technology, for the purpose of the description of certain embodiments. These figures may not precisely reflect the characteristics of any given embodiment, and are not necessarily intended to define or limit specific embodiments within the scope of this technology.

### DETAILED DESCRIPTION

The following description of technology is merely exemplary in nature of the subject matter, manufacture, and use of one or more inventions, and is not intended to limit the scope, application, or uses of any specific invention claimed in this application or in such other applications as may be filed claiming priority to this application, or patents issuing therefrom. A non-limiting discussion of terms and phrases intended to aid understanding of the present technology is provided at the end of this Detailed Description.

The present technology relates to knee revision and knee arthroplasty implant components, as well as methods for improving the strength and usefulness of medical implants. It is envisioned that the medical implant can be one of various orthopedic implants, for example, a femoral component or femoral assembly; a knee revision; a total knee arthroplasty, and the like. The medical implant can be a standard size, custom made, or a deformable generic shape for filling in a bone defect caused by surgical intervention or disease. As referenced herein, the term “implant” may be used to refer to an entire implant, or a portion thereof; portions may be as large or as small as necessary to accommodate the specific need. For example, an implant made in accordance with the present technology, generally including a polymer body portion with a reinforcement member secured to at least one of the lateral and medial condylar portions thereof, may constitute the entire implant, or it may be used with one or more additional pieces or components that together form a final implant or implant assembly. As such, the present technology encompasses a wide variety of therapeutic and cosmetic applications, in human or other animal subjects. It should be understood that the specific materials and devices used must be biomedically acceptable. As used herein, such a “biomedically acceptable” component is one that is suitable for use with humans and/or animals without undue adverse side effects (such as toxicity, irritation, and allergic response) commensurate with a reasonable benefit risk/ratio.

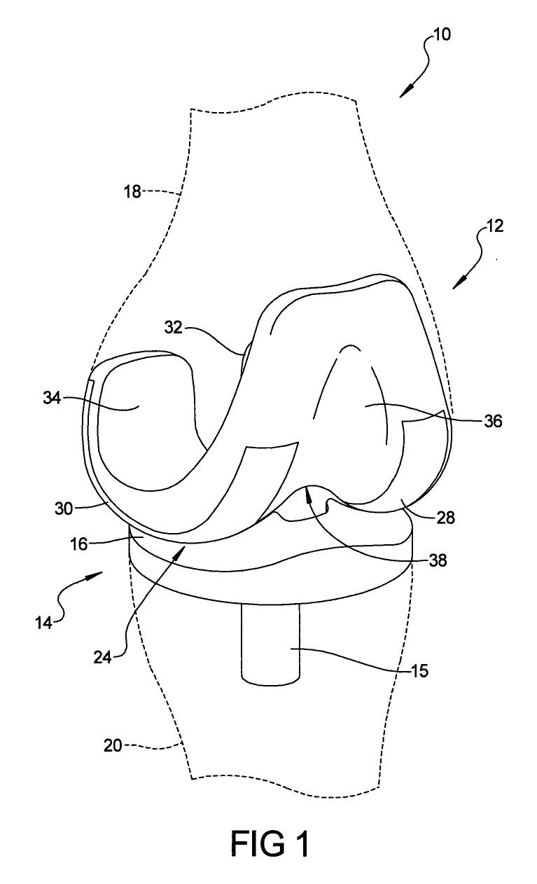

In various aspects, the present technology provides an improved femoral component for a knee joint prosthesis including at least one reinforced articulating surface. FIG. 1 generally illustrates an exemplary knee joint prosthesis assembly 10 that can include a femoral component 12 for attachment to a femur 18 and a tibial component 14 for attachment to a tibia 20. The knee joint prosthesis 10 is generally shown to include a tibial component 14 that can support a bearing 16 that engages cooperating articulation surfaces 24, 26 of the femoral component 12. The tibial component 14 can have a bone engaging inferior surface and a bearing engaging superior surface. The tibial component 14 can be unitary or modular in construction and generally can include a stem portion 15 that may be used to connect to a tibial tray/bearing portion 16 as is known in the art. Such a tray or bearing 16 can be integrally or modularly coupled to the tibial component 14. Various adapter assemblies may be also provided as known in the art. In various aspects, the femoral component 12 can be formed or shaped from a lightweight and high strength polymer, as will be discussed below. The tibial component 14, as well as the tray or bearing 16 may also be formed of a high strength polymer, or a biocompatible metal, ceramic, thermoplastic, or combination thereof as known in the art.

It should be understood that the various aspects of the present technology can be used with knee joint prostheses having various designs, both standardized and custom made, and may include numerous design variations as known in the art. The present technology can be readily used with the Vanguard Complete Knee System (VCKS) manufactured by Biomet Manufacturing Corp. of Warsaw, Ind. Additional description of this system, as well as various other designs, configurations, geometries, stems, adaptors, couplings, trays, bearings, and related components, etc. can be found in commonly owned and U.S. patent application Ser. No. 11/972,359, filed on Jan. 10, 2008 and published as U.S. Pub. No. 2008/0167722; U.S. patent application Ser. No. 12/248,517, filed on Oct. 9, 2008 and published as U.S. Pub. No. 2009/0125114; U.S. patent application Ser. No. 12/536,056, filed on Aug. 5, 2009 and published as U.S. Pub. No. 2009/0299482; and U.S. patent application Ser. No. 12/788,961, filed on May 10, 2010 and published as U.S. Pub. No. 2010/0305710, each of which is incorporated by reference in its entirety.

It is envisioned that the respective components of a knee prosthesis assembly 10 of the present disclosure can be patient specific, such that each component can be constructed for optimal features for a given patient. By way of example, the bone interface margins of the femoral component 12 and tibial component 14 can be patient specific for optimized bone coverage. In addition, the overall size, such as anterior-posterior dimensions and bone cut geometry can be determined and used for manufacturing the components of the knee prosthesis assembly 10. Some articulation features can also be determined and used as criteria for forming the components of the knee prosthesis assembly 10.

Thus, each of the various components of the knee prosthesis assembly 10 can be a patient-specific or custom-made implant, a semi-custom implant, an off-the-shelf implant, or otherwise standard production implant. A patient-specific or custom-made implant is essentially a one-of-a-kind implant specifically made for a particular patient, and consequently there is no inventory associated with such implant. Standard, or off-the shelf, implants are typically available and stocked in a number of sizes, typically six or more, or ten or more, with a number of configurations or types, including bilateral or unilateral implants, constrained, semi-constrained, mobile, etc. Because of the variety of sizes and configurations that are kept in stock to be accommodated by different patients, a large inventory of standard implants may be created, and several molds for each type and size of implant may be used. Semi-custom implants can provide an intermediate solution between custom-made and off-the-shelf implants. Semi-custom implants reduce the size of inventory and molds required for production, while allowing some degree of patient-specific customization.

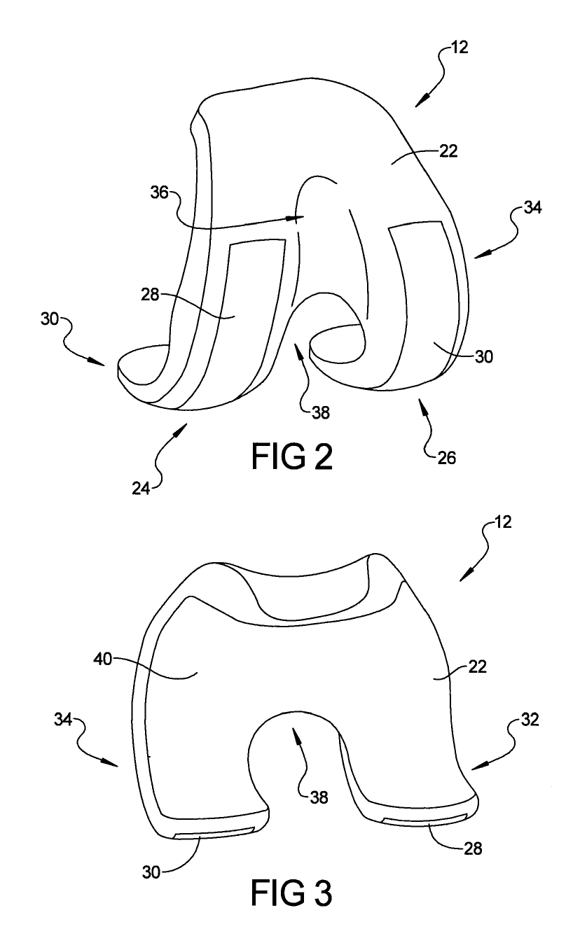

With reference to FIGS. 1-3, various aspects of the present technology provide a femoral component 12 having a polymer body portion 22 with reinforced articulating or bearing surfaces 24, 26 for articulation with substantially complementary bearing surfaces of the tibial 14 component or tibial bearing 16. As will be described, the polymer body 22 may be provided with rigid reinforcement members, or inserts 28, 30 formed with, inserted, or secured to the articulating or bearing surfaces 24, 26. There may be a plurality of various sized and dimensioned pre-manufactured polymer femoral components 12 available for use. In certain aspects, and where applicable, each of the plurality of pre-manufactured polymer femoral components 12 can be provided with a similar bearing surface geometry and dimensions such that they are each configured to mate with a standardized size reinforcement member 28, 30. The femoral component 12 can include a medial condyle portion 32 and a lateral condyle portion 34. The condyle portions 32, 34 can replace the medial and lateral condyles of a distal femur 18. In this regard, the femoral component 12 can be any of a patient-specific or custom-made implant, a semi-custom implant, an off-the-shelf implant, or otherwise standard production implant, while there may only be a need to manufacture a limited number, for example one to three, of different sized reinforcement members 28, 30.

The present technology provides a lightweight femoral component 12 having a polymer body portion 22. It can be formed from any suitable surgical grade, low friction, and low wearing biocompatible polymer or plastic, such as polyethylene, and in particular, ultra-high molecular weight polyethylene (UHMWPE), polyetheretherketone (PEEK), or other suitable material. In certain aspects, the polymer femoral component comprises an injection molded plastic. By using injection molding techniques, the reinforcement members 28, 30 can be molded directly within the polymer body portion 22, which may provide superior and more permanent fastening. In other aspects, the polymer femoral component can be formed using direct compression molding (DCM) techniques. For example, DCM techniques provide extremely smooth surface finishes, and the reinforcement members 28, 30 can be secured within the component for a net-shape product.

The polymer femoral component 12 can generally comprise a cruciate retaining prosthesis and can include various portions to replace or mimic the distal femur 18. As shown, the medial and lateral condyle portions 32, 34 can interconnect and the polymer femoral component 12 can be formed as a single piece with an intercondylar anterior portion (or patellar track) 36. The intercondylar anterior portion 36 can allow for articulation of a patella, either a natural or prosthetic patella, once the femoral component 12 is implanted onto the distal femur 18. The medial and lateral condyle portions 32, 34 and the intercondylar anterior portion 36 can generally define an exterior portion of the femoral component 12. The femoral component 12 can define an opening or passage 38 between the medial and lateral condyle portions 32, 34. As can be appreciated, the passage 38 can accommodate, and provide clearance for a host ACL and/or PCL or a reconstructed ACL and/or PCL. The medial and lateral condyle portions 32, 34 can include a substantially spherical contact surface that is convex in an anterior/posterior direction and a medial/lateral direction. In various aspects, a metal patella may be provided operable to articulate with the polymer intercondylar anterior portion 36 of the femoral component 12. The femoral component 12 can include a bone contacting or inferior surface 40 that can be substantially flat (FIG. 3).

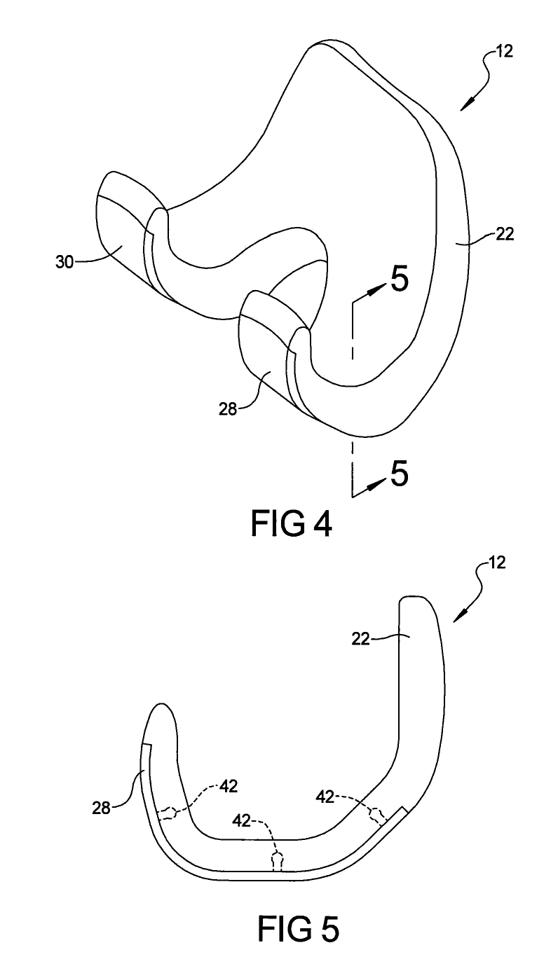

The present technology provides for rigid condylar reinforcement members 28, 30 that can be inserted into, press-fit, or integrally formed within the polymer body portion 22 in order to provide reinforced articulating or bearing surfaces 24, 26. FIG. 4 is a perspective posterior view of a femoral component; FIG. 5 is a cross sectional view of the femoral component of FIG. 4, taken along the line 5-5. The specific types of insertion or attachment techniques may vary, and the reinforcement members 28, 30 can be provided with various fastening means as known in the art. For example, the reinforcement members 28, 30 can include stems 42 or anchors as shown in FIG. 5. The polymer body 22 can be provided with cooperating apertures to engage the stems 42. The reinforcements 28, 30 may be also secured using dovetail joints, t-shaped slots, or the like. While in most instances the attachment will be permanent, it is envisioned that there may be applications where the fastening means are provided such that the reinforcement members 28, 30 are removably fastened. In other aspects, the reinforcement members 28, 30 can be attached to the polymer body 22 using locking grooves (not shown) or known mechanical fasteners, such as screws.

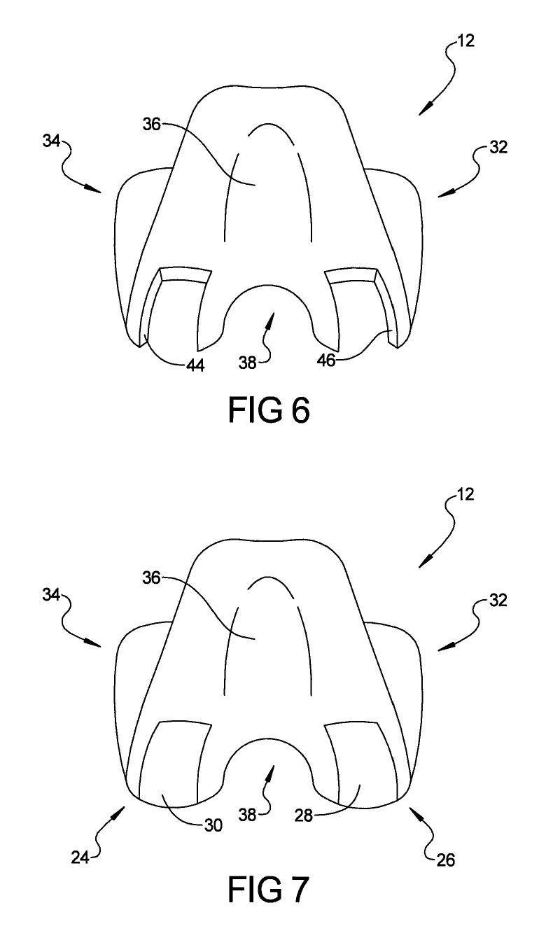

With reference to FIGS. 6 and 7, in various aspects, the polymer body portion 22 can be molded or otherwise formed having internal recesses 44, 46 defined therein, configured for receiving the reinforcement members 28, 30 that cooperate to form the reinforced articulating bearing surfaces 24, 26. In other aspects, internal recesses 44, 46 can be shaped and defined using sculpting tools as known in the art. In certain aspects, the rigid reinforcing members 28, 30 are at least partially disposed within the internal recesses 44, 46. In other aspects, the rigid reinforcing members 28, 30 are completely disposed within the internal recesses 44, 46, providing a smooth reinforced bearing surface.

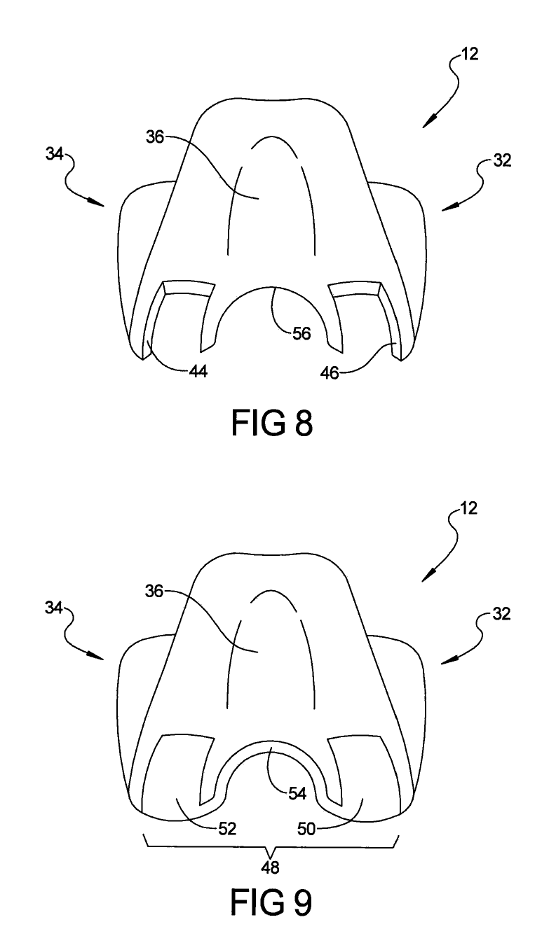

With reference to FIGS. 8 and 9, in certain aspects, the reinforcement members may be formed as a unitary component 48 having multiple regions or portions. For example, the unitary reinforcement member 48 may be provided with a medial portion 50 and a lateral portion 52 that are coupled together by a bridge portion 54. As shown in FIG. 8, the polymer body 22 can be provided with condyle recesses 44, 46 as well as an additional bridge portion recess area 56. For example, the internal recess area can extend from the lateral condylar portion bearing surface region, across the lower part of the intercondylar anterior portion, and to the medial condylar bearing surface region. The bridge portion 54 can be appropriately sized to provide sufficient strength while minimizing any additional weight. The bridge portion 54 may have a size and a thickness commensurate with the remainder of the unitary reinforcement member 48, or it may be provided with a variable size, as necessary. In various aspects, it is envisioned that the rigid reinforcement member 48 can be provided with standardized dimensions such that it is operable to mate with any one of a plurality of various sized pre-manufactured polymer femoral components 22.

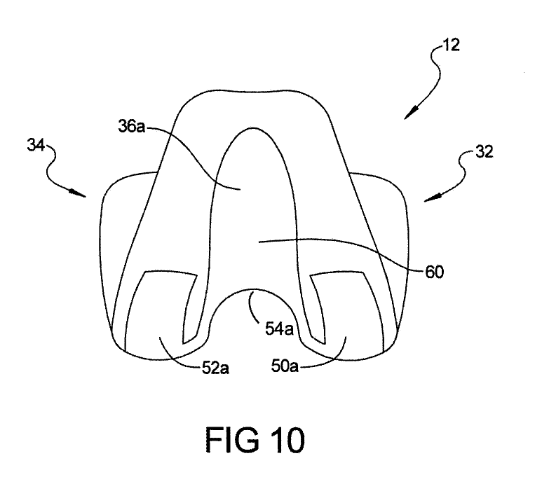

FIG. 10 provides still another aspect of the reinforcement member formed as a unitary component 60 having multiple regions or portions. For example, the unitary reinforcement member 60 may be provided with a medial portion 50a and a lateral portion 52a that are coupled together by a large bridge portion 54a that extends a distance covering a substantial portion of the intercondylar anterior portion 36a. In this aspect, the internal recess area can extend from the lateral condylar portion bearing surface region, across the middle to lower part of the intercondylar anterior portion, and to the medial condylar bearing surface region. It should be understood that a plastic patella would be used if the unitary reinforcement member 60 with the bridge portion 54a extending a distance up the intercondylar portion 36a is provided as a metal. The bridge portion 54a extending up the intercondylar anterior portion 36a can be appropriately sized with a thickness that provides sufficient strength while minimizing any additional weight. For example, the thickness of the intercondylar anterior portion 36a may be commensurate with the remainder of the unitary reinforcement member 60, or it may be provided having a slightly reduced thickness to lower the weight of this non-weight bearing surface.

In certain aspects, the reinforcement members 28, 30, 48, 60 can be formed of a biocompatible metal material such as a solid metal, porous metal, or a combination of solid metal and porous metal. In one example, the solid metal or porous metal can comprise stainless steel, titanium, titanium alloys, tantalum, tungsten, cobalt, chromium, molybdenum, cobalt-chromium alloys such as a CoCrMo alloy, and other materials, combinations, or alloys thereof that are suited for use in a biocompatible environment.

In other aspects, the reinforcement members 28, 30, 48, 60 can be formed of a rigid thermoplastic material, such as polyetheretherketone (PEEK) or carbon reinforced PEEK.

In still other aspects, the rigid condylar reinforcement members 28, 30, 48, 60 can be formed of a ceramic material or ceramic composition. Ceramics used in the implants of the present technology can be made of any suitable biomedically acceptable ceramic material. Generally, such ceramic materials include inorganic, non-metallic materials that are processed or consolidated at a high temperature. Suitable ceramic materials may include structural ceramics, as opposed to ceramic powders that are resorbable, for example, hydroxyapatite and calcium phosphate. Suitable structural ceramic materials may include one or more ceramic oxides or non-oxides, such as carbides, borides, nitrides, sulfides, and silicides. Particular oxides may include alumina and zirconia as well as titanium oxide and titanium dioxide. Zirconia can be chemically “stabilized” in several different forms, including magnesia-stabilized zirconia, calcium oxide stabilized zirconia, and yttria-stabilized zirconia. Particular non-oxides may include silicon nitride and silicon carbide, or metal nitride and metal carbide. Doped ceramics may also be used, such as yitria, magnesium oxide, strontium oxide, alumina, and combinations thereof. In certain aspects, a ceramic composition may include small ceramic particles, optionally including a ceramic powder, disposed within certain of the spaces or voids between relatively larger ceramic fragments. In various aspects, such smaller particles or powder may be provided of selected quantity and having selected size and shape, so as to create and to adjust an overall porosity or to increase the strength and stabilization, as desired. Ceramic fragments, if used, may have random overall shapes, sizes, and geometries. Alternatively, or in addition, ceramic compositions used with the present technology may further comprise reinforcing materials, for example metal filler. Suitable metals may include one or more of tantalum, gold, tungsten, cobalt, chromium, titanium, and alloys thereof. The metal filler can be present in various shapes such as randomly shaped particles, spherical powder, fibers, whiskers, rods, or random shapes.

With each combination of material selections, the polymer body portion 22 will typically have a first hardness, and the reinforcement members 28, 30, 48, 60 can have a second hardness. In order to provide the proper reinforcement, the second hardness is greater than the first hardness. When separate lateral and medial reinforcement members 28, 30 are used, in certain aspects they may be provided having a substantially similar articulating geometry such that they are interchangeable with one another. In other aspects, the reinforcement members 28, 30 may need to have different or custom geometries. The reinforcement members 28, 30 may be provided with a substantially uniform thickness of from about 1 mm to about 12 mm, or greater as necessary. It may be desirable for certain reinforcement members to be provided with a reduced thickness; for example, less than 5 mm, from about 1 mm to about 4 mm, or from about 2 mm to about 3 mm. Depending on the strength or other properties of the material, in other aspects, the reinforcement members may be provided with an increased thickness; for example, greater than 5 mm, from about 8 mm to about 11 mm, or from about 9 mm to about 10 mm. It is envisioned that the bridge portions 54, 54a and any area covering the intercondylar region 36a may be provided with a reduced thickness as compared to the weight bearing surface areas.

In various aspects, the components of the knee joint prostheses as described herein can be provided in kits. For example, a kit may include a plurality of femoral components of various sizes, with two or more different types of rigid reinforcement members 28, 30, 48, 60. It is envisioned that the rigid reinforcement members 28, 30, 48, 60 can be of a standard size to correspond to a single, articulating geometry of the various sized femoral components. In other aspects, the reinforcement members 28, 30, 48, 60 may be provided having a range of outer arcuate radii at the bearing surfaces to match custom or specific patient geometries.

The embodiments described herein are exemplary and not intended to be limiting in describing the full scope of compositions and methods of the present technology. Equivalent changes, modifications and variations of embodiments, materials, compositions and methods can be made within the scope of the present technology, with substantially similar results.

### Non-limiting Discussion of Terminology:

The headings (such as “Introduction” and “Summary”) and sub-headings used herein are intended only for general organization of topics within the present disclosure, and are not intended to limit the disclosure of the technology or any aspect thereof. In particular, subject matter disclosed in the “Introduction” may include novel technology and may not constitute a recitation of prior art. Subject matter disclosed in the “Summary” is not an exhaustive or complete disclosure of the entire scope of the technology or any embodiments thereof. Classification or discussion of a material within a section of this specification as having a particular utility is made for convenience, and no inference should be drawn that the material must necessarily or solely function in accordance with its classification herein when it is used in any given composition.

The description and specific examples, while indicating embodiments of the technology, are intended for purposes of illustration only and are not intended to limit the scope of the technology. Moreover, recitation of multiple embodiments having stated features is not intended to exclude other embodiments having additional features, or other embodiments incorporating different combinations of the stated features. Specific examples are provided for illustrative purposes of how to make and use the compositions and methods of this technology and, unless explicitly stated otherwise, are not intended to be a representation that given embodiments of this technology have, or have not, been made or tested.

As used herein, the words “desire” or “desirable” refer to embodiments of the technology that afford certain benefits, under certain circumstances. However, other embodiments may also be desirable, under the same or other circumstances. Furthermore, the recitation of one or more desired embodiments does not imply that other embodiments are not useful, and is not intended to exclude other embodiments from the scope of the technology.

As used herein, the word “include,” and its variants, is intended to be non-limiting, such that recitation of items in a list is not to the exclusion of other like items that may also be useful in the materials, compositions, devices, and methods of this technology. Similarly, the terms “can” and “may” and their variants are intended to be non-limiting, such that recitation that an embodiment can or may comprise certain elements or features does not exclude other embodiments of the present technology that do not contain those elements or features.

Although the open-ended term “comprising,” as a synonym of non-restrictive terms such as including, containing, or having, is used herein to describe and claim embodiments of the present technology, embodiments may alternatively be described using more limiting terms such as “consisting of” or “consisting essentially of.” Thus, for any given embodiment reciting materials, components or process steps, the present technology also specifically includes embodiments consisting of, or consisting essentially of, such materials, components or processes excluding additional materials, components or processes (for consisting of) and excluding additional materials, components or processes affecting the significant properties of the embodiment (for consisting essentially of), even though such additional materials, components or processes are not explicitly recited in this application. For example, recitation of a composition or process reciting elements A, B and C specifically envisions embodiments consisting of, and consisting essentially of, A, B and C, excluding an element D that may be recited in the art, even though element D is not explicitly described as being excluded herein.

When an element or layer is referred to as being “on”, “engaged to”, “connected to” or “coupled to” another element or layer, it may be directly on, engaged, connected or coupled to the other element or layer, or intervening elements or layers may be present. In contrast, when an element is referred to as being “directly on”, “directly engaged to”, “directly connected to” or “directly coupled to” another element or layer, there may be no intervening elements or layers present. Other words used to describe the relationship between elements should be interpreted in a like fashion (e.g., “between” versus “directly between,” “adjacent” versus “directly adjacent,” etc.).

What is claimed is:

1. A femoral component for a knee joint prosthesis, the femoral component comprising:
a polymer body having first and second condylar portions interconnected by an intercondylar anterior portion;
a first reinforcement member secured to the first condylar portion; and
a second reinforcement member secured to the second condylar portion.

2. The femoral component of claim 1, wherein the reinforcement members comprise a material selected from the group consisting of a metal alloy, a ceramic material, and a thermoplastic material.

3. The femoral component of claim 2, wherein the reinforcement members comprise a CoCrMo alloy.

4. The femoral component of claim 1, wherein the first and second reinforcement members are formed as a unitary component, coupled together by a bridge portion.

5. The femoral component of claim 4, wherein the bridge portion extends a distance covering the intercondylar anterior portion.

6. The femoral component of claim 1, wherein the polymer body has a first hardness and the reinforcement members comprise a rigid thermoplastic material having a second hardness that is greater than the first hardness.

7. The femoral component of claim 6, wherein the polymer body comprises polyethylene and the reinforcement members comprise polyetheretherketone (PEEK).

8. The femoral component of claim 1, wherein polymer body comprises a direct compression molded plastic wherein the reinforcement members are molded within the polymer body.

9. The femoral component of claim 1, wherein the first and second reinforcement members comprise a substantially similar articulating geometry and are interchangeable with one another.

10. The femoral component of claim 1, wherein the first and second reinforcement members comprise a substantially uniform thickness of from about 2 mm to about 3 mm.

11. The femoral component of claim 1, wherein each condylar portion defines an internal recess, and the reinforcement members are secured within the respective recesses providing each condylar portion a smooth, reinforced articulating bearing surface.

12. The femoral component of claim 11, wherein the reinforcement members are removably fastened within the respective condylar portion internal recesses.

13. A knee joint prosthesis, comprising:
a polymer femoral component including:
an intercondylar anterior portion;
first and second condylar portions extending from the intercondylar anterior portion;
a first rigid reinforcement insert secured to the first condylar portion, defining a first reinforced bearing surface;
a second rigid reinforcement insert secured to the second condylar portion, defining a second reinforced bearing surface; and
a tibial component having a bone engaging inferior surface and a bearing engaging superior surface configured to engage the first and second bearing surfaces of the polymer femoral component.

14. The knee joint prosthesis of claim 13, wherein the first and second reinforcement inserts comprise a material selected from the group consisting of a metal alloy, a ceramic material, and a thermoplastic material.

15. The knee joint prosthesis of claim 13, wherein the first and second reinforcement inserts are formed as a unitary component, coupled together by a bridge portion.

16. The knee joint prosthesis of claim 15, wherein the bridge portion extends a distance covering the intercondylar anterior portion.

17. The knee joint prosthesis of claim 13, further comprising a metal patella component operable to articulate with the intercondylar anterior portion of the polymer femoral component.

18. The knee joint prosthesis of claim 13, wherein the polymer femoral component has a first hardness and the rigid reinforcement inserts have a second hardness that is greater than the first hardness.

19. The knee joint prosthesis of claim 13, wherein the first and second condylar portions define internal recesses, and the first and second rigid reinforcement inserts are press-fit into the respective recesses.

20. A knee joint prosthesis assembly, comprising:
a polymer femoral component including at least one of a lateral and medial condylar portion having a bearing surface extending from an intercondylar anterior portion and defining an internal recess;
a rigid reinforcement member disposed within the internal recess; and
a tibial component having a bearing surface that is complementary to the at least one condylar bearing surface of the polymer femoral component.

21. The knee joint prosthesis of claim 20, wherein polymer femoral component comprises both a lateral condylar portion and a medial condylar portion, each having a respective bearing surface, and the internal recess extends from the lateral condylar portion across the intercondylar anterior portion and to the medial condylar portion.

22. The knee joint prosthesis of claim 20, wherein the rigid reinforcement member is provided with standardized dimensions operable to mate with any one of a plurality of various sized pre-manufactured polymer femoral components.
